(19) United States
(12) Patent Application Publication
(10) Pub. No.: US 2004/0259088 A1
(43) Pub. Date: Dec. 23, 2004

(54) Method for analyzing RNA using time of flight secondary ion mass spectrometry

(75) Inventors: Tadashi Okamoto, Kanagawa (JP); Hiromitsu Takase, Tochigi (JP); Hiroyuki Hashimoto, Kanagawa (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, TOKYO (JP)

(21) Appl. No.: 10/463,574

(22) Filed: Jun. 18, 2003

(30) Foreign Application Priority Data
Jun. 28, 2002 (JP) 189838/2002

(51) Int. Cl. C12Q 1/68

(52) U.S. Cl. 435/6

(57) ABSTRACT

The analysis method according to the present invention provides a method for detecting the target nucleic acid in the sample, in which the problems caused by employing the radio isotope and fluorescent methods can be solved, thereby enabling the acquisition of gene information with higher accuracy. A method for analyzing a target nucleic acid in a sample is conducted by: reacting the sample with a probe support having two or more probes fixed thereon, which contain a base portion being complementary with a base sequence of the target nucleic acid; and detecting an existence of a hybridized complex of the probe and the target nucleic acid using time of flight secondary ion mass spectrometry, in which the hybridized complex is formed when the target nucleic acid is contained in the sample, wherein the target nucleic acid and the nucleic acid probe are a combination of RNA and DNA.

### BACKGROUND OF THE INVENTION

1. Field of the Invention

The present invention relates to an analysis of RNA or DNA that are gene related materials.

2. Description of the Related Art

A nucleic acid chip such as DNA chip, RNA chip and so on has been employed for the purposes of analyzing genome or analyzing generation of gene, and it is expected that the result of the analysis thereof provides critical index for diagnosis of cancers, gene diseases, life style-related diseases, infection diseases and the like, prediction for prognostics, or decision of treatment policy and so on.

Several methods for preparing the above-described nucleic acid chips are known. On describing the methods for preparing a DNA chip as examples, the exemplary methods for preparing a DNA chip may include: a method of consecutively synthesizing DNA probes on a substrate by using photolithography (U.S. Pat. No. 5,405,783 and so on); or a method for supplying synthesized DNA or synthesized cDNA (complementary DNA) onto a substrate and being bound thereto (U.S. Pat. No. 5,601,980, Japanese Patent Laid-Open No. 11-187,900 (1999), an article from “SCIENCE”, Vol. 270, pp. 467 (1995) and so on).

In anyway, nucleic acid chips can be prepared in accordance with these methods, and a target nucleic acids can be analyzed to eventually acquire the desired gene information by: leaving the prepared nucleic acid chip in a hybridization condition within a solution containing the target nucleic acids; detecting whether the hybridization of the obtained nucleic acid probe and the target nucleic acid is found or not with any means; and further analyzing thereof. In this occasion, the nucleic acid probe on the biochip is principally presented as a single molecular film level, and the amount of the target nucleic acid forming the hybridized complex therein may sometimes vary small in some cases, as the amount of the target nucleic acid also depends on the concentration of the target nucleic acid in the solution containing the target nucleic acid. Therefore, the means for detecting the above-mentioned hybridized complex requires the means of very high sensitivity, and the conventional examples of such means may include the combination of the radioisotope labeling to the target nucleic acid and autoradiography, or the combination of fluorescent labeling to the target nucleic acid and the fluorescence detector such as fluorescent scanner.

However, in these conventional examples, the combination using the radioisotope is not a common method, since the procedure is complicated, and dangerous and/or special equipment and/or apparatus are required. The combination using the fluorescence is often employed, since the procedure thereof is relatively simple and the sensitivity thereof is high, but the method may involve several problems in the aspect of quantification-ability and reproducibility such as insufficient chemical stability and quenching of the fluorescent dye, nonspecific adsorption of the fluorescent dye onto the substrate surface and the like, as well known. Other general high sensitivity-surface analysis methods include ATR method that utilizes FT-IR method (Fourier Transform Infra Red Spectrometry), XPS method (X-ray Photoelectron Spectrometry) and so on. However, these methods do not involve sufficient sensitivity for the quantitative analysis of the probe of the nucleic acid chip. In particular, when a general purpose glass is employed as a substrate for the nucleic acid chip, these methods are not useful analysis methods, since the absorption due to the glass substrate itself adversely affects the analysis results when FT-IR (ATR) method is employed for example, or since the charge-up occurred on the glass adversely affects the analysis results when XPS method is employed.

Another high sensitivity-surface analysis method may be a DNA detection method utilizing laser RIS (Resonance Ionization Spectroscopy) method, which is disclosed in U.S. Pat. No. 5,821,060. In this method, the specimen surface is irradiated with a laser beam having a wavelength that is equivalent to ionization energy of a specific element, so that the specific element is ionized and released from the specimen surface and the released ionized element is detected, and disclosed methods for releasing the element from the specimen surface may be a method utilizing laser beam or a method utilizing ion. However, these methods have a technical limitation in which only limited elements are possible to be detected. Yet another high sensitivity-surface analysis method may be dynamic SIMS (Secondary Ion Mass Spectrometry), in which an organic compound is decomposed to smaller fragment ions or to particles during the process of generating secondary ion, and thus, the amount of the information on the chemical structures obtained from the mass spectrum is poor, and thus the method is not suitable for the use in the analysis of organic compounds such as nucleic acid-related materials.

On the other hand, the time of flight secondary ion mass spectrometry (TOF-SIMS), which is also known as another technique of the secondary ion mass spectrometry, is an analysis method for investigating what types of atoms or molecules are existing on the uppermost surface of a solid specimen, and the method has the advantages described below: having a detection ability for detecting trace amount of a component of 10⁹ atoms/cm² (equivalent to 1/10⁵ of the all atoms existing in one atomic layer of the uppermost surface); being applicable to both organic and inorganic compounds; being capable of detecting all types of elements and compounds existing on the surface; and being available of imaging secondary ions from materials existing on the surface of the specimen.

Here, the principles of the time of flight secondary ion mass spectrometry will be described as follows. In high vacuum condition, a high-speed ion beam (primary ion) applied to a surface of a solid specimen causes sputtering phenomenon, in which a structural components of the surface are released into the vacuum. Ions (secondary ions) having positive or negative charges generated during this occasion are converged to a direction by applying an electrical field, and then the ions are detected at a position that is far therefrom by a constant distance. In the sputtering process, various ions having variety of masses are generated depending on the chemical components of the surface of the specimen, and the lighter ions fly faster and, on the contrary, heavier ions fly slower, within a constant electrical field, and thus, detecting the time taken from the generation of the secondary ions to the arrival of the generated ions to the detector (i.e., time of flight) provides an analysis of the mass of the generated secondary ions.

In the conventional dynamic-SIMS method, organic compounds are decomposed to fragment ions or particles during the ionization process as stated above, and thus information on the chemical structure obtained from the mass spectrum is poor. On the contrary, in the TOF-SIMS method, the structures of the organic compounds can be obtainable from the mass spectrum measurements, since the extremely smaller amount of the applied primary ions is necessary in the TOF-SIMS method, so that the organic compounds are ionized with substantially retaining their chemical structure. The information on the uppermost layer (within a depth of several angstroms) of the object can be obtainable as only the secondary ions generated in the outermost portions of the solid object surface are released into the vacuum.

An exemplary example of detecting the nucleic acid from a single molecular film that is fixed to the substrate via TOF-SIMS method is reported (Proceeding of the 12^{th} International Conference on Secondary Ion Mass Spectrometry, 951 (1999)), and in this example, decomposed fragment ions of bases and decomposed fragment ions of phosphate backbone are described as exemplary nucleic acid fragment ions that can be detected via TOF-SIMS.

### SUMMARY OF THE INVENTION

However, when one desires to obtain desirable gene information by detecting the target DNA by using a DNA chip, which is a commonly used procedure, with TOF-SIMS method as a detection method, it is often impossible to specifically detect the existence of the hybridized complex of the target DNA, for the two reasons of:

(1) The detectable portion for TOF-SIMS method is limited to the portion of the very thin layer near the surface: and

(2) The fragment ion species generated by the target DNA is identical to the fragment ion species generated by the probe DNA, thereby causing problems in the detection process.

One of the methods for solving the problem may be a method of combining PNA (peptide nucleic acid) to a solid phase to form a probe, thereby forming a hybridized complex with the target nucleic acid. (J. C. Feldner et al., SIM XIII International Conference, Nov. 11-16, 2001, Nara, Japan) According to this method, since peptide base have a base portion identical to DNA but have no phosphate backbone, formation of the hybridized complex of PNA probe with target nucleic acid is confirmed if the fragment ions derived by the phosphate backbone.

However, since peptide nucleic acid is expensive, the acquisition of gene information by using peptide nucleic acid for probe may often cause higher cost, and thus such acquisition may not be practical in many occasions.

A method for analyzing a target nucleic acid in a sample according to the present invention is conducted by: reacting the sample with a probe support having two or more probes fixed thereon, which contain a base portion being complementary with a base sequence of the target nucleic acid; and detecting an existence of a hybridized complex of the probe and the target nucleic acid using time of flight secondary ion mass spectrometry, in which the hybridized complex is formed when the target nucleic acid is contained in the sample, wherein a combination of the target nucleic acid and the nucleic acid probe is a combination of RNA and DNA.

The analysis method according to the present invention provides a method for detecting the target nucleic acid in the sample, in which the aforementioned problems caused by employing the radio isotope method and fluorescent method can be solved, thereby enabling the acquisition of gene information with higher accuracy.

Further objects, features and advantages of the present invention will become apparent from the following description of the preferred embodiments with reference to the attached drawings.

### BRIEF DESCRIPTION OF THE DRAWINGS

FIG. 1 shows results of the imaging conducted in Example 2, in which FIG. 1-A represents the results using PO₂⁻ ion, and FIG. 1-B represents the results using (adenine-H)⁻ ion.

FIG. 2 shows another result of the imaging conducted in Example 2, representing the results using (uracil-H)⁻ ion.

### DETAILED DESCRIPTION OF THE PREFERRED EMBODIMENTS

A probe fixed on a support according to the present invention is specifically combinable with a specific target material. DNA or RNA is employed for the probe of the present invention. DNA available for the present invention may include genome DNA and cDNA (complementary DNA), and oligonucleotides and/or polydeoxynucleotides that are synthesized to have a specific sequence. RNA available for the present invention, which is synthesized to have a specific sequence, may include oligoribonucleotides.

An example of the probe that is supported on the support may be a probe containing a bonding portion to the support, optionally via a linker therebetween, included in a part of oligonucleotide composed of a base sequence that can be hybridized with the target nucleic acid, and the bonding portion to the support has a structure of bonding to the surface of the support. Here, the position of the bonding portion to the support in the oligonucleotide molecule is not particularly limited as long as the selected position is not adversely affect the desired hybridization reaction.

Here, a probe support is defined as a support having a plurality of probes fixed on a respective area on the surface of the support, that is for example, respective fixing area for respective probe is designed to be a dot-like spot, and also a probe array is defined as an array of the probes in an arrangement disposing probes at equal pitch. Further, the array of the fixing area at higher density corresponds to a microarray. In addition, the probe support includes a nucleic acid chip such as DNA chip, RNA chip and the like.

On the other hand, the probe has a structure of being combinable with the support surface, and the fixing of the probe onto the support surface may preferably be conducted via the structure of being combinable with the support surface. In such configuration, the structure of being combinable with the support surface may preferably be formed by a processing of introducing at least one organic functional group such as amino group, thiol group, carboxyl group, hydroxy group, acid halides (haloformyl group: —COX), halides (—X), aziridine, maleimide group, succinimide group, isothiocyanate group, sulfonyl chloride group (—SO₂C1), aldehyde group (formyl group: —CHO), hydrazine, acetamide iodide and so on. Further, the fixing of the probe via covalent bond can be achieved by conducting a processing that is required for treating the support surface depending upon the structure in the probe required for forming the binding of the probe to the support, that is for example, a processing of forming on the support surface a functional group, e.g., maleimide group for thiol group as the structure in the probe or epoxy group, aldehyde group or N-hydroxy succinimide group for amino group as the structure in the probe. Here, the probe may preferably be bound to the substrate surface via covalent bond, in view of achieving better chemical stability.

According to the present invention, the formation of the hybridized complex is can be confirmed by detecting a fragment ion that is specific to a target nucleic acid using time of flight secondary mass spectrometry. The fragment ion may be suitably selected by combining the probe to be used and the target nucleic acid. When the probe is DNA and the target nucleic acid is RNA, (uracil-H)⁻ ion may be selected for an index for the detection. That is, RNA does not contain thymine, one of four DNA bases, but instead contains uracil, so that if (uracil-H)⁻ ion is detected as the fragment ion that is specific to RNA, it is confirmed that a hybridized complex of DNA probe and target RNA is formed thereon.

RNA for the use in the present invention may be any RNA as long as RNA can be detected and analyzed via TOF-SIMS and is available for the use in desired analysis methods. When RNA is mRNA (messenger RNA), transcribed gene information can be obtained as it is. When RNA is tRNA (transfer RNA) or rRNA (ribosomal RNA), although gene information to be translated for the synthesis of protein can not be obtained, information of tRNA or rRNA themselves is obtainable.

When target nucleic acid is DNA, (thymine-H)⁻ ion, which is specific to DNA, may be selected for a fragment ion, and a formation of a hybridized complex is confirmed by detecting the fragment ion using time of flight secondary ion mass spectrometry. DNA available as target nucleic acid for the present invention may include, for example, genome DNA and cDNA (complementary DNA). RNA that is preferably available as a probe in this case may include oligoribonucleotides and polyribonucleotides.

Target DNA for the use in the present invention may be any DNA as long as DNA can be detected and analyzed via TOF-SIMS and is available for the use in desired analysis methods. When target DNA is genome DNA, gene information of genome can be directly obtainable, and when target DNA is cDNA (complementary DNA), gene information transcribed to mRNA can be indirectly known. In addition, DNA obtained by amplifying genome DNA or cDNA via PCR (Polymerase Chain Reaction) can also be employed as target DNA.

Each of the unit processes used in the method for preparing the probe support employed in the present invention can be conducted by a known procedure. Depending on the case, the probe may be prepared by being sequentially synthesized on the support surface, or the probe may be synthesized in advance and then the synthesized probe may be supplied onto the support surface.

In this occasion, the ink jet method may be preferably employed for supplying the probe onto the support surface, since employing the ink jet method provides production of fine probe support with higher density. The available ink-jet methods may include the known piezo-jet method and the thermal jet method.

### EXAMPLES

The present invention will be described specifically, by illustrating examples. In the following examples, the respective processing of handling RNA was carried out in the RNase-free condition.

### (Example 1)

### Preparation of a DNA Probe Chip by Using dT40 Probe

A DNA probe chip was prepared in accordance with a known method (i.e., a method described in the Japanese Patent Laid-Open No. 11-187,900 (1999)).

(1) Washing of the Substrate

A synthesized quartz substrate having a dimension of 25.4 mm×25.4 mm×1 mm was disposed in a rack, and the substrate was immersed in a detergent solution that contains a detergent for ultrasonic washing (GPIII, commercially available from BRANSON) diluted to 10% with pure water for one night. Then, the substrate was ultrasonic-washed in the detergent solution for 20 minutes, and after that the substrate was washed with water to remove the detergent. After rinsed with pure water, the substrate was further ultrasonic-washed within a container containing pure water for 20 minutes. Next, the substrate was immersed in aqueous solution of 1N sodium hydroxide that was pre-heated to 80° C., for 10 minutes. Sequentially, the substrate was washed with water and further washed with pure water, and was transferred to the next unit processing as it was.

(2) Surface Treatment

An aqueous solution of 1% wt. of N-β-(aminoethyl)-γ-aminopropyltrimethoxysilane, KBM603 (commercially available from SHIN-ETSU CHEMICAL IND. CO. LTD.), which is a silane coupling agent having amino acids bonded thereto, was stirred at room temperature for 2 hours to achieve hydrolysis of methoxy group contained in the molecular of the above-mentioned silane compound. The washed substrate that was washed in the process described in the above section (1) was then immersed into the aqueous solution of the silane coupling agent for 1 hour, and after that the substrate was washed with pure water, and the both sides of the substrate was dried by being blown with nitrogen gas to the both sides. Next, the substrate was baked in an oven that was heated to 120° C., for 1 hour, and thereby amino acids were eventually introduced onto the surface of the substrate. Next, 2.7 mg of N-(Maleimidocaproyloxy)succinimide (commercially available from DOJINDO LABORATORIES, hereinafter called “EMCS”) was dissolved into a solution of 1:1 (by volumetric ratio) of dimethyl sulfoxide (DMSO)/ethanol to prepare a solution having a concentration of 0.3 mg/ml. The substrate, which had been treated via silane-coupling treatment, was immersed in the EMCS solution at room temperature for 2 hours to react the amino group, which is introduced to the substrate surface via the silane coupling treatment, with the succinimide group of EMCS. In this stage, maleimide group that was derived from EMCS existed on the substrate surface. The substrate was then picked up from the EMCS solution, was washed with the mixed solvent of DMSO and ethanol, was sequentially washed with ethanol, and then was dried by being blown with nitrogen gas.

(3) Synthesis of Probe DNA

Single strand nucleic acid of sequence 1 (40mer of dA) was synthesized, by ordering DNA synthesis company (BEX CO. LTD.). Thiol group (SH) was introduced to the 5′ end of the single strand DNA of the sequence 1, by using thiol modifier (available from GLENN RESEARCH CENTER) during the synthesis. Here, the deprotecting and the recovering of DNA were carried out according to the ordinary methods, and DNA was purified by using HPLC. The series of the processing from the synthesis to the purification was conducted by the aforementioned DNA synthesis company.

Sequence 1 (sequence number: 1)
5′HS—(CH₂)₆-O—PO₂-O—AAAAAAAAAA AAAAAAAAAA
AAAAAAAAAA AAAAAAAAAA 3′

(4) DNA Discharge by Using a Thermal Jet Printer and Binding of DNA to the Substrate

The single strand DNA of the above sequence 1 was dissolved into an solution, which contained 7.5% wt. of glycerin, 7.5% wt. of urea, 7.5% wt. of thioglycol, and 1% wt. of acetylene alcohol (under the product name of “ACETYLENOL EH”, commercially available from KAWAKEN FINE CHEMICAL CO., LTD.), at a concentration of 8 μM. A printer head (“BC-50”, commercially available from CANON CO. LTD.) for a bubble jet printer (“BJF-850”, commercially available from CANON CO. LTD.), which employs a bubble jet method that is one of the thermal jet methods, was altered so that the altered printer head was capable of discharging several-hundred μl of the solution. The altered printer head was mounted to a discharge drawing device, which was also altered so as to be capable of discharging the solution onto the flat quartz substrate. Several-hundred μl of the above-mentioned DNA solution was transferred into an altered tank of the printer head, and the EMCS-treated substrate was mounted to the discharge drawing device, carrying out a spotting operation onto the EMCS-treated surface of the substrate. Here, the discharge rate during the spotting operation was 4 pl/droplet, the area of the spotting operation was 10 mm×10 mm disposed around the center of the substrate, and the spotting was carried out at 200 dpi for that area, i.e., the discharge was performed at a pitch of 127 μm. In this condition, the diameter of the spotted dot was approximately 50 μm.

After completing the spotting operation, the substrate was left in a humidifier chamber for 30 minutes so that maleimide group of the glass plate surface was reacted with thiol group of the end of the nucleic acid probe, thereby fixing the DNA probe thereon. Then, the substrate was washed with pure water, and stored in the pure water

### (Example 2)

### Detection and Analysis of Hybridized Complex via TOF-SIMS

(1) Synthesis of a Model Target RNA

A model target RNA of sequence 2(40mer of U: uracil) was synthesized, by ordering a DNA synthesis company (BEX CO. LTD.), as similarly in Example 1.

Sequence 2 (sequence number: 2)
5′ UUUUUUUUUU UUUUUUUUUU UUUUUUUUUU UUUUUUUUUU 3′

(2) Blocking

Prior to carry out a hybridization of the DNA chip prepared in Example 1 with the above-described model target RNA, a blocking was conducted by using BSA (bovine serum albumin, commercially available from SIGMA ALDRICH JAPAN), for the purpose of preventing a nonspecific adsorption onto the surface of the target RNA. More specifically, BSA was dissolved into 50 mM phosphate buffer solution (pH=7.0) containing 1M NaOH at a concentration of 2%, and the DNA chip was immersed in the resultant solution at a room temperature for 3 hours, and then, after rinsed with the above-described phosphate buffer solution, the following hybridization was conducted.

(3) Hybridization

RNA of 40mer of U was dissolved in the above-described phosphate buffer solution at a concentration of 50 nM, and then the DNA chip that was treated with the blocking treatment was included in 2 ml of the resultant solution (contained in a Hybri-pack), to carry out the hybridization process at 45° C. for 15 hours. After that, the chip was rinsed with the above-described phosphate buffer solution and then, after rinsed with pure water at room temperature, the chip was dried by being blown with nitrogen gas, and was stored in a vacuum desiccator.

(4) Analysis via TOF-SIMS

DNA chip processed by hybridization was analyzed via TOF-SIMS. Here, the apparatus used for carrying out the analysis was “TOF-SIMS IV”, commercially available from ION TOF. In addition, the semi-processed chip, which was treated until the blocking process but not treated with hybridization, was also analyzed as a control. The apparatus conditions were listed below.

<Primary Ion>

primary ion beam: 25 kV Ga⁺, random scan mode;

pulse frequency of the primary ion beam: 2.5 kHz (400 μsec./shot);

pulse width of the primary ion beam: 1 ns; and

beam diameter of the primary ion beam: 5 μm.

<Secondary Ion: Imaging was Carried out by Reconstructing the Obtained Data According to the Application Pattern of the Primary Ion Beam>

detection mode for secondary ion: negative;

area for the measurement: 300 μm×300 μm;

number of pixel in the secondary ion image: 128×128 pixels; and

number of integrating operation: 256.

(5) Measurement Results

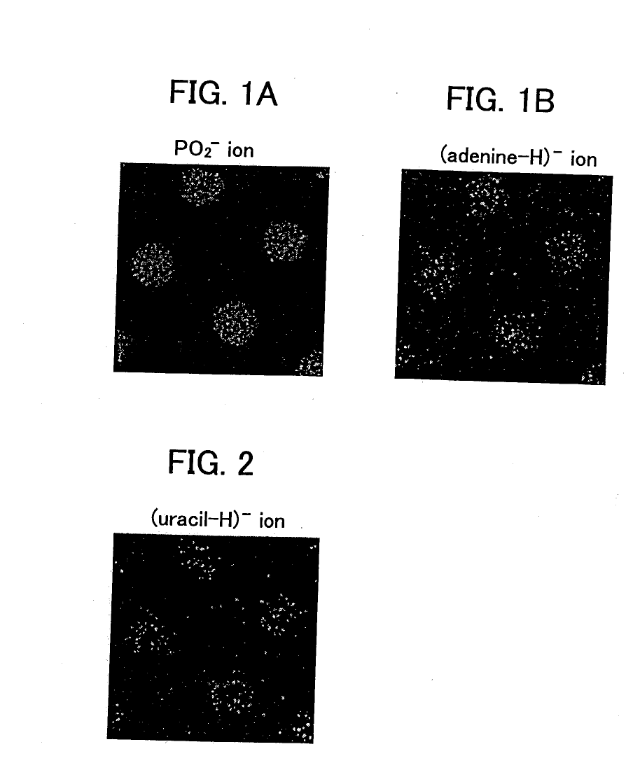

First, a part of the measurement results of the semi-processed chip, which was treated until the blocking process but not treated with hybridization and employed as a control, are shown. In general, decomposed fragment ions are commonly detected in the case of analysis of DNA and RNA via TOF-SIMS as stated above, and FIG. 1-A represents the result of imaging (analysis), which shows that DNA from the DNA chip is combined thereto in dotted form, by using PO₂⁻ (m/z=63) as one of the decomposed fragment ions. FIG. 1-B shows the result of imaging identical portion by using (adenine-H)⁻ ion (m/z=134). Other fragment ions derived by other nucleic acid bases including (uracil-H)⁻ ion was not detected. These results indicate that the DNA probes consisting of adenylic acid were formed in a dotted form on the prepared DNA chip, as expected.

FIG. 2 represents the result of imaging of the DNA chip, which was prepared by conducting hybridization with 40mer of U by using (uracil-H)⁻ ion (m/z=110). It can be seen therefrom that uracil is included in the dotted portion. The aforementioned PO₂⁻ (m/z=63) and (adenine-H)⁻ ion were also detected from the dotted potion, and according to these results, it is confirmed that DNA of the chip and the target RNA form hybridized complex.

### (Example 3)

### Preparation of RNA Chip by Using U40 Probe, and Hybridization and Analysis via TOF-SIMS Thereof.

Sequence 3
5′HS—(CH₂)₆-O—PO₂-O—UUUUUUUUUU UUUUUUUUUU
UUUUUUUUUU UUUUUUUUUU 3′
Sequence 4
5′ AAAAAAAAAA AAAAAAAAAA AAAAAAAAAA AAAAAAAAAA 3′

RNA chip was prepared in the similar procedure to Example 1 by using RNA of sequence 3 (U40mer) obtained from the DNA synthesis company. Then, hybridization of the RNA chip and a target DNA of sequence 4 (dA40mer), which was also obtained from the synthesis company, was carried out, and analysis was conducted via TOF-SIMS. The results shows that (adenine-H)⁻ ion was detected only at the dotted portions in which the hybridization was conducted. According to the results, it is confirmed that RNA of the chip and the target DNA form hybridized complex.

While the present invention has been described with reference to what are presently considered to be the preferred embodiments, it is to be understood that the invention is not limited to the disclosed embodiments. On the contrary, the invention is intended to cover various modifications and equivalent arrangements included within the spirit and scope of the appended claims. The scope of the following claims is to be accorded the broadest interpretation so as to encompass all such modifications and equivalent structures and functions.

What is claimed is:

1. A method for analyzing a target nucleic acid in a sample by: reacting the sample with a probe support having two or more probes fixed thereon, said probe containing a base portion being complementary with a base sequence of said target nucleic acid; and detecting an existence of a hybridized complex of the probe and the target nucleic acid using time of flight secondary ion mass spectrometry, said hybridized complex being formed when the target nucleic acid is contained in said sample, wherein
a combination of said target nucleic acid and said nucleic acid probe is a combination of RNA and DNA.

2. The method according to claim 1, wherein a fragment ion, which is specific to, said target nucleic acid is detected using time of flight secondary ion mass spectrometry method.

3. The method according to claim 2, wherein said target nucleic acid is RNA and said fragment ion is (uracil-H)⁻ ion.

4. The method according to claim 1, wherein said RNA is mRNA.

5. The method according to claim 1, wherein said RNA is tRNA.

6. The method according to claim 1, wherein said RNA is rRNA.

7. The method according to claim 3, wherein said nucleic acid probe is DNA, which is bonded to a surface of said support via covalent bond.

8. The method according to claim 7, wherein said DNA is polydeoxynucleotide.

9. The method according to claim 3, wherein said DNA is cDNA.

10. The method according to claim 2, wherein said target nucleic acid is DNA, and said fragment ion is (thymine-H)⁻ ion.

11. The method according to claim 1, wherein said DNA is genome DNA.

12. The method according to claim 1, wherein said DNA is cDNA.

13. The method according to claim 10, wherein said nucleic acid probe is RNA, which is bonded to a surface of said support via covalent bond.

14. The method according to claim 13, wherein said DNA is oligoribonucleotides.


SEQUENCE LISTING (2 sequences)

SEQ ID NO: 1 - DNA, 40 bp, Artificial Sequence
  Sequence for Hybridization Test
aaaaaaaaaaaaaaaaaaaaaaaaaaaaaaaaaaaaaaaa

SEQ ID NO: 2 - RNA, 40 nt, Artificial Sequence
  Sequence for Hybridization Test
uuuuuuuuuuuuuuuuuuuuuuuuuuuuuuuuuuuuuuuu